(19) United States
(12) Patent Application Publication
(10) Pub. No.: US 2011/0236308 A1
(43) Pub. Date: Sep. 29, 2011

(54) Aryl Bidentate Isonitriles and Their Uses

(75) Inventors: David R. Elmaleh, Newton, MA (US); Timothy M. Shoup, Waltham, MA (US); Alan J. Fischman, Boston, MA (US); Bhaskar C. Das, Cambridge, MA (US)

(73) Assignee: The General Hospital Corporation, Boston, MA (US)

(21) Appl. No.: 12/297,513

(22) PCT Filed: Apr. 17, 2007
(86) PCT No.: PCT/US07/09450

§ 371 Date: Jun. 16, 2011

Related U.S. Application Data
(60) Provisional application No. 60/793,032, filed on Apr. 19, 2006.

(51) Int. Cl. A61K 51/04; C07C 255/33; C07C 255/51; C07F 13/00; A61K 47/48; A61P 43/00

(52) U.S. Cl. 424/1.65; 558/302; 534/10; 534/14; 514/525

(57) ABSTRACT

The present invention relates generally to chelation compounds, radionuclide metal chelate compounds, i.e., complexes, and radiolabeled targeting moieties, i.e., conjugates, formed therefrom, and methods of using these compounds, complexes and conjugates for diagnostic and therapeutic purposes. This invention is more particularly related to aryl bidentate isonitrile metal complexes generated by a chemical interaction of a metal salt oxide or weak complex of the metal with aryl isonitrile bidentate ligands.

### CROSS-REFERENCE TO RELATED APPLICATIONS

This application claims benefit of U.S. provisional application No. 60/793,032, filed Apr. 19, 2006, the content of which is incorporated herein by reference in its entirety.

### FIELD OF THE INVENTION

The present invention relates generally to chelation compounds, radionuclide metal chelate compounds, i.e., complexes, and radiolabeled targeting moieties, i.e., conjugates, formed therefrom, and methods of using these compounds, complexes and conjugates for diagnostic and therapeutic purposes. This invention is more particularly related to aryl bidentate isonitrile metal complexes generated by a chemical interaction of a metal salt oxide or weak complex of the metal with aryl isonitrile bidentate ligands.

### BACKGROUND OF THE INVENTION

The ability to obtain in vivo images has assisted in treatment, diagnosis and prognosis of a variety of diseases and disorders. A range of imaging agents, for example radioimaging agents, have been developed, but have suffered from problems such as cost, complexity, and the need to identify specific ligands that can be used to deliver desired radionuclides.

A limitation of current diagnostic imaging methods is that it is often not possible to deliver the imaging agent specifically to the organ or tissue that one wishes to image. In the case of target organ imaging, what is needed is an agent that is specific for the target organ, yet does not exhibit appreciable uptake in non-target organs.

Various radioactive metals (radionuclides) have been prepared including Tc, Ru, Co, Pt, Fe, Os, Ir, W, Re, Cr, Mo, Mn, Ni, Rh, Pd, Nb and Ta, see e.g., U.S. Pat. No. 4,452,774; U.S. Pat. No. 4,826,961, U.S. Pat. No. 5,783,170; U.S. Pat. No. 5,807,537; U.S. Pat. No. 5,814,297; U.S. Pat. No. 5,866,097; and U.S. Patent application 2002187099. However, in order to effectively deliver such radionuclides one needs to prepare coordination complexes with ligands. The specific coordination requirements of particular radionuclides place constraints on the ligands that can be used, which in turn place limits on what are viable targets. Ideally, a radionuclide imaging complex should display specific targeting in the absence of substantial uptake in non-target tissues or organs, and a capacity for targeting to the desired targets. Thus, there still exists a need in the art for methods to develop and achieve effective delivery of imaging agents to target sites such as tumors by simple and general means.

Coronary heart disease (CHD) is the leading cause of death in the United States, accounting for roughly 30% of all deaths. The cost of cardiovascular disease in 2002 was estimated by the American Heart Association at $330 billion. This figure covers direct costs, which include the cost of physicians and other professionals, hospital and nursing home services, the cost of medications, home health care and lost productivity resulting from morbidity and mortality. More than 50% of emergency room chest pain patients are admitted to hospitals unnecessarily at an estimated annual cost of $12 billion. A recent study with Tc-99m sestamibi (CARDIOLITE®, Bristol-Myers Squibb Medical Imaging, Inc.) suggest that a convenient test that assesses the myocardium can help emergency room physicians better identify which patients may or may not be having a heart attack or near heart attack. It concluded that when the test was incorporated into ER evaluation, there was a 20% reduction in unnecessary hospitalizations per year. Hence, there is a need for sensitive, reliable, and low cost techniques for early detection of heart disease and for monitoring the course of treatment.

The table 1 tabulates the SPECT imaging agents typically used at present:

TABLE 1

| FDA approved | Investigational |
|---|---|
| Thallium-201 | ^{99m}Technetium-furifosmin |
| ^{99m}Technetium-sestamibi ( [symbol] Cardiolite) | |
| ^{99m}Technetium-tetrofosmin ( [symbol] Myoview) | ^{99m}Technetium-NOET |
| ^{99m}Technetium-teberoxime ( [symbol] Cardiotec) | |

All of these agents are designed to be given to a patient as an intravenous injection, be extracted and trapped by the myocardium or tissue in question in proportion to blood flow. The agents differ by their pharmacokinetics: accumulation, trapping and washout and the representation of flow.

All of the existing agents have similar degree of diagnostic ability. However most of them are not optimized for their sensitivity. In normal myocardium blood flow, appearance at rest and during exercise shows homogenous uptake and distribution. In patients with ischemic heart disease and pronounced vessel restriction exercise or adenosine blunt the regional heart image in the compromised tissue. The efficacy of these imaging procedures is not optimized in most instances where heart disease is early and there is limited vessel constriction. Most nuclear cardiac imaging is done using single photon emission computed tomography (SPECT). This technique is able to record perfusion images of about ½ cm thick three-dimensional slices. SPECT images of the myocardium can differentiate between scared myocardium and ischemic myocardium and teach about ventricular function using the rest and stress exercise. Improving flow representation can increase the reliability of the diagnosis and the follow up of therapy.

Hence, there is a need for sensitive, reliable, and low cost techniques for early detection of heart disease and for monitoring the course of treatment. These agents will improve the sensitivity and accuracy of the test when imaged with SPECT and with other metal complexes.

### BRIEF DESCRIPTION OF THE DRAWINGS

FIGS. 1A-1I describe the chemical structures of some of the embodiments of the aryl bidentate isonitriles of the invention and the prior art. FIG. 1A shows sestamibi (CARDIOLITE®). FIG. 1B shows the Tc-99m complex of the structure in FIG. 1E. FIG. 1C shows a novel F-18 blood flow agent developed in our group. FIGS. 1E, 1F and 1G show examples of aryl bidentate ligands used for Tc-99m chelation of the present invention. FIG. 1H shows an example general aryl substitution on FIG. 1E for controlling charge size and lipohilicity. FIG. 1I shows a potential structure of the complex prepared from the structure of FIG. 1H.

FIG. 2 shows sequential images obtained from the Tc-99m complex generated from the structure in FIG. 1G in a rat 10-60 minutes post-injection.

FIG. 3 shows single photon emission computed tomographic (SPECT) serial slices of the left ventricle in the short axis (top two panels), vertical long axis (mid two panels), and horizontal long axis (lower two panels) planes. A 3-D model of the heart is also shown for orientation. The short axis slices begin at the apex (left corner) and end at the base (right corner). The vertical long axis slices begin at the septum (left corner) and end at the lateral wall (right corner), and the horizontal long axis slices begin at the inferior wall (left corner) and end at the anterior wall (right corner). For each tomographic plane, the stress images are displayed at the top and the rest or delayed images at the bottom. The images show normal tracer distribution and were obtained during exercise in a 35 year old man with a low probability of coronary artery disease.

### DESCRIPTION OF THE INVENTION

The invention relates to aryl bidentate isonitrile ligands and the metal complexes produced by a chemical interaction of a metal salt oxide or weak complek of the metal with the aryl isonitrile bidentate ligands. Isonitrile complexes were reported in the literature in the early nineties and several patents claimed their use with radionuclides such as Tc-99m. Sestamibi is a Tc-99m complex, produced from six methoxyisobutyl isonitrile groups and a reduced form of Tc-99m, which became a commercial product used in myocardial imaging with Single Photon Emission Computed Tomography (SPECT). Bidentate isonitriles were mentioned by Jones, Davison, and Abrams (U.S. Pat. No. 4,452,774), but the ligands actually made were all alkyls. Furthermore, later applications directed to isonitrile complexes focused on modified alkyls. Aryl bidentate isonitriles were never part of the molecular design nor synthesized by any group. We have discovered a novel series of bidentate aryl isonitriles with a unique chemical structure. The Tc-99m complexes formed with theses new ligands show pronounced myocardial uptake in an animal rat model. The structure of these aryl bidentate ligands allows control over lipophilicity and charge that improve uptake performance and help mimic the behavior of chemical microspheres. Additionally, one can vary the substitutions to tailor other characteristics of the complex such as what is the target tissue, circulating half-life, etc.

In certain embodiments, the compounds of the invention comprise a series of novel aryl bidentate isonitrile ligands, where the isonitrile function is in close proximity to the aryl moiety to allow for better charge distribution over the molecule. In another embodiment, the positive charge is distributed over the molecule improving its lipophilicity and its penetration of cell membranes. In certain embodiments, the bidentate moieties are more remote enhancing the positive charge effect of the molecule.

Another aspect of the invention relates to imaging different tissues such as the myocardium or thyroid or specific disease such as tumors or carcinomas.

In one preferred embodiment the agent complexed to the aryl bidentate isonitrile ligands is a radionuclide. Another aspect of the invention relates to the aforementioned compounds that is attached to a fluorescent agent. Another aspect of the invention relates to compounds comprising aryl bidentate isonitrile metal complexes and pharmaceutical agents covalently bound to the metal complex. In certain embodiments, the pharmaceutical agents include, but are not limited to, antisense, protein, liposome, siRNA, antibiotic, antiviral, a fluorescent molecule, MRI contrast metal or radionuclide. Another aspect of the present invention relates to a method of treating disease involving administering the compounds of the invention to a mammal. Another aspect of the present invention relates to methods of acquiring optical, magnetic resonance or radionuclide imaging using the metal compounds of the invention.

The present invention provides novel aryl bidentate isonitriles and diagnostic kits thereof. Radiolabeled complexes of these isonitriles, e.g., Tc-99m, and diagnostic methods utilizing the radiolabeled complexes.

More specifically, one aspect of this invention provides novel aryl isonitriles (and kits thereof) of the structure:

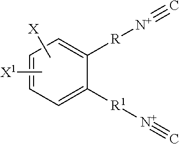

Wherein,

R and R1 each independently is a lower straight or branched chain alkyl, alkenyl, alkynyl, fluoroalkyl, halo alkenyl, acyl, alkoxyalkyl, ester, keto, or aldehyde group linking the nitrogen atom of the isonitrile group to the aryl group. Preferably, about 0 to about 4 carbons separate the nitrogen atom of the isonitrile group from the carbon of the aryl group; and

X and X1 each independently is a hydrogen or a substituent selected from the group consisting of alkyl, alkenyl, alkynyl, fluoroalkyl and haloalkenyl having up to 12 carbon atoms; halo; carbamoyl; amido; amino; acyl; acyloxy; cyano; alkoxy; alkoxyalkyl; ester groups; keto groups; and aldehyde groups, or pharmaceutically acceptable salts thereof. By varying the substituents, the practitioner of the invention can control the charge size, lipophilicity and stability of the complex.

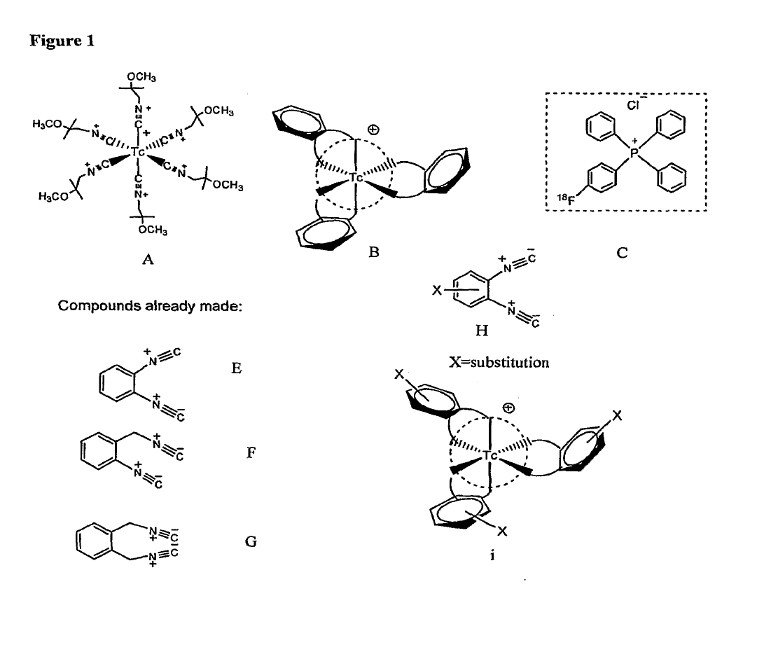

The invention includes three aryl bidentate isonitriles bound to a central radionuclide or metal, as depicted in FIGS. 1B and 1I.

Another aspect of the invention relates to novel metal complexes of these aryl bidentate isonitrile ligands, that may also be represented by the general formula:

Z(CN—R—ArX₀₋₂—R¹—NC)₃

Three aryl bidentate isonitriles bond to Z. In one preferred embodiment, Z is a radionuclide selected from radioactive isotopes of Tc, Ru, Co, Pt, Fe, Os, Ir, W, Re, Cr, Mo, Mn, Ni, Rh, Pd, Nb, and Ta, for example, 99 mTc, 99Tc, 97Ru, 51Cr, 57Co, 188Re and 191Os. In a particularly preferred embodiment, Z is 99 mTc. Alternatively, Z is a fluorescent agent. Alternatively, Z is a metal wherein the metal is covalently bound to a pharmaceutical agent, for example, a nucleic acid, e.g., siRNA, an antibiotic, an antiviral, a fluorescent molecule, an MRI contrast agent or a radionuclide.

Any desired counterion can be present as required by the charge on the complex with the proviso that such counterion must be pharmaceutically acceptable if the complex is to be used in vivo.

Generally, aryl bidentate isonitrile radionuclide complexes are prepared by procedures which introduce the radionuclide at a late stage of the synthesis. This allows for maximum radiochemical yields, and reduces the handling time of radioactive materials. When dealing with short half-life isotopes, a major consideration is the time required to conduct synthetic procedures, and purification methods. Protocols for the synthesis of radiopharmaceuticals are described in Tubis and Wolf, Eds., “Radiopharmacy”, Wiley-Interscience, New York (1976); Wolf, Christman, Fowler, Lambrecht, “Synthesis of Radiopharmaceuticals and Labeled Compounds Using Short-Lived Isotopes”, in Radiopharmaceuticals and Labeled Compounds, Vol 1, p. 345-381 (1973), the disclosures of each of which are hereby incorporated herein by reference, in their entirety.

The complexes of the present invention can easily be prepared by admixing a salt of the radioactive metal and the aryl bidentate isonitrile ligand in the presence of a suitable reducing agent, if required, in aqueous media at temperatures from room temperature to reflux temperature or even higher, and are obtained and isolatable in high yield at both macro (carrier added, e.g. 99Tc) concentrations and at tracer (no carrier added, e.g. 99 mTc) concentrations of less than 10-6 molar. In some cases the aryl bidentate isonitrile ligand can itself act as the reducing agent thus eliminating the need for an additional reducing agent. Suitable additional reducing agents, when required or desired are well known to those skilled in the art. The reaction is generally complete after 5 minutes to 2 hours, depending upon the identity of the particular reagents employed. The radiolabelled complex is made in the same way as the corresponding non-radioactive aryl bidentate isonitrile complex by simply substituting the desired radionuclide for the corresponding non-radioactive element in the starting materials, except in the case of technetium because all technetium isotopes are radioactive.

In the case of a radionuclide, such as, for example, 99 mTc, a complex in accord with this invention is preferably made by mixing pertechnetate (Tc-7) with the desired aryl bidentate isonitrile in aqueous medium, then adding to the reaction mixture an appropriate reducing agent capable of reducing the technetium. Among suitable reducing agents are alkali metal dithionites, stannous salts, sodium borohydride, and others, as is well known.

The isonitrile technetium complexes of this invention can also be prepared from preformed technetium complexes having oxidation states for technetium of, for instance, +3, +4 or +5, by treating these preformed complexes with an excess of isonitrile ligands under suitable conditions. For example, the technetium-aryl bidentate isonitrile complex can also be prepared by reacting the desired aryl bidentate isonitrile ligand with the hexakis-thiourea complex of Tc+3 or with a technetium-glucoheptonate complex, or the like.

An excess of the aryl bidentate isonitrile ligand, up to 50 to 100% molar excess or more, and an excess of reducing agent, can be used in the complexing reaction to ensure maximum yield from the technetium. Following the reaction, the desired complex can be separated from the reaction mixture, if required, by crystallization or precipitation or by conventional chromatography or ion exchange chromatography.

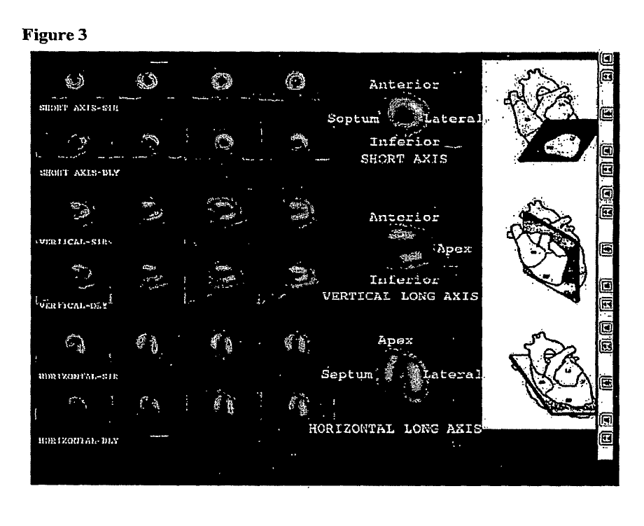

The complexes of this invention are useful in visualizing cardiac tissue, as shown in FIG. 3. In alternative embodiments, the compositions can be used to image tissues other than heart, e.g., detecting the presence of thrombi in the lung and associated areas of blood perfusion deficits, imaging organs and functions including bone marrow, the hepatobiliary system, metastatic tumor deposits in the liver, pulmonary perfusion and pulmonary thromboembolic disease, renal perfusion and excretory function, skeletal muscle perfusion and abnormalities of both skeletal muscle and myocardial energetics as may occur in cardiomyopathies and diseases of mitochondrial dysfunction such as mitochondrial cytopathies or “ragged red fiber” disease. The complexes are further useful for radioactive tagging of cells and formed elements of blood, other animal cells, plant cells, and small organisms which possess membranous exteriors, e.g., single-cell entities, microbes, etc. In addition, they can be employed to label previously prepared liposomes without the necessity for encapsulation as is the case with many other labeling agents. The complexes can also be employed therapeutically.

The choice of radionuclides will depend on the use. For example, preferred radionuclides for diagnostic imaging are radioactive isotopes of Tc, Ru, Co, Pt, Fe, Os, and Ir; preferred radionuclides for therapeutic uses are radioactive isotopes of W, Re, Fe, and Os; preferred radionuclides for radioactive tagging are Cr, Mo, Co, Tc, Fe, Mn, W, Ru, Ni, Rh, Ir, Pd, Nb, and Ta.

For diagnostic purposes Tc-99m is the preferred isotope. Its 6 hour half-life and 140 keV gamma ray emission energy are ideal for gamma scintigraphy using equipment and procedures well established for those skilled in the art. The rhenium isotopes also have gamma ray emission energies that are compatible with gamma scintigraphy, however, they also emit high energy beta particles that are more damaging to living tissues. However, these beta particle emissions can be utilized for therapeutic purposes, for example, cancer radiotherapy, and thus may be utilized in the composition and methods of the present invention for combination diagnostic and therapeutic purposes.

The technetium radionuclides are preferably in the chemical form of pertechnetate or perrhenate and a pharmaceutically acceptable cation. The pertechnetate salt form is preferably sodium pertechnetate such as obtained from commercial Tc-99m generators. The amount of pertechnetate used to prepare the radiopharmaceuticals of the present invention can range from 0.1 mCi to 1 Ci, or more preferably from 1 to 200 mCi.

The aryl bidentate isonitrile complexes of the present invention are administered to an individual via methods known to those of skill in the art for administering radionuclide imaging agents. The particular dosage employed need only be high enough to obtain diagnostically useful images, generally in the range of 0.1 to 20 mCi/70 Kg body weight.

Administration of a composition may be by systemic route, including oral, parenteral, sublingual, rectal such as suppository or enteral administration, or by pulmonary absorption. Parenteral administration may be by intravenous injection, subcutaneous injection, intramuscular injection, intra-arterial injection, intrathecal injection, intra peritoneal injection or direct injection or other administration to one or more specific sites.

Access to the gastrointestinal tract, which can also rapidly introduce substances to the blood stream, can be gained using oral enema, or injectable forms of administration. Compositions may be administered as a bolus injection or spray, or administered sequentially over time (episodically) such as every two, four, six or eight hours.

The invention further provides methods of administering the aryl bidentate isonitrile radionuclide complexes to an individual comprising the steps of: preparing a aryl bidentate isonitrile radionuclide complex according to the methods of the invention and administering an effective amount of the aryl bidentate isonitrile radionuclide complex to said individual. The aryl bidentate isonitrile radionuclide complexes of the invention may be administered intravenously, intraarterially, intranasally such as by aerosol administration, nebulization, inhalation, or insufflation, intratracheally, intra-articularly, orally, transdermally, subcutaneously. Methods of administration for amphipathic compounds are equally amenable to administration of compounds that are insoluble in aqueous solutions.

In one embodiment, aryl bidentate isonitrile ligands complexed with the desired agent, e.g., radionuclide, are administered to individuals for diagnostic purposes. In this embodiment, the individual is imaged at a time point known to those of skill in the art and dependant on the particular radionuclide used, e.g. after the aryl bidentate isonitrile complex has entered all tissues, bound to a target cell, and non-bound aryl bidentate isonitrile complexes have cleared sufficiently so that there is a target to background differential. This process allows for optimal background to signal ratios and for technetium-99m is at least 2 hours; preferably 6 hours, but may also be 3, 4, 5, 6, 7, 8, 9, 10, 11, 12, or more hours. The time will further vary depending on the radionuclide used.

Also encompassed in the present invention are kits for preparing the aryl bidentate isonitrile complexes of the present invention. Kits in accord with the present invention comprise a quantity of a reducing agent for reducing a preselected radionuclide. Preferably, such kits contain a predetermined quantity of an aryl bidentate isonitrile ligand and a predetermined quantity of a reducing agent capable of reducing a predetermined quantity of a preselected radionuclide. It is also preferred that the aryl bidentate isonitrile ligand and reducing agent be lyophilized, when possible, to facilitate storage stability. If lyophilization is not practical, the kits are stored frozen. The aryl bidentate isonitrile ligand and reducing agent are generally provided in sealed, sterilized containers.

In one embodiment of the invention, a kit for use in making the complexes of the present invention from a supply of 99 mTc such as the pertechnetate solution in isotonic saline available in most clinical laboratories includes the desired quantity of a selected aryl bidentate isonitrile ligand to react with a selected quantity of pertechnetate, and a reducing agent such as sodium dithionite or stannous chloride in an amount sufficient to reduce the selected quantity of pertechnetate to form the desired complex.

A molar excess of the aryl bidentate isonitrile ligand, typically 600% molar excess or more, and an excess of reducing agent, can be used in the complexing reaction to ensure maximum yield of the desired complex from the technetium. Following the reaction, the desired complex can be separated from the reaction mixture, if required, by crystallization or preCipitation or by conventional chromatography or ion exchange chromatography.

In another embodiment, the compositions of the present invention may be used to treat disease by the enhancement of tissue uptake of therapeutic pharmaceuticals. For example, tumor therapy using cytotoxic agents directed at tumor mitochondria could be enhanced as could the cellular toxicity of chemotherapeutic agents. Along this line, enhanced tumor uptake of radiation sensitizing agents could be promoted.

In an alternative embodiment of the present invention, lipophilic cationic aryl bidentate isonitrile complexes of paramagnetic metal ions such as Gd, Dys, Fe, or Mn can be coadministered with agents that decrease the intramembrane potential. Complexes of paramagnetic metal ions produce relaxation enhancement of tissues placed within a strong magnetic field. Since relaxation enhancement is proportional to the local concentration of the paramagnetic metal complex, tissue relaxation differences can be augmented by use of the present invention during diagnostic tests with magnetic resonance imaging technology.

In another alternative embodiment of the present invention, lipophilic cation aryl bidentate isonitrile complexes of rhenium can be co-administered with agents that decrease the intramembrane potential. Because rhenium can produce ionizing radiation in sufficient local quantities to serve as a therapeutic radiopharmaceutical, the present invention can enhance tissue accumulation of the agent and improve its potential use in tumor therapy.

Radiosensitizing or radioprotectant compositions may be administered along with the radionuclide aryl bidentate isonitrile compositions of the present invention. Radiosensitizing agents work by increasing the singlet oxygen species in the vicinity of the target and therefore increasing its radiosensitivity. Other compounds used in conjunction with radiation therapy include radioprotectants which are designed to protect surrounding tissue from some of the effects of radiation therapy.

Thus, in addition to radionuclides, pharmacologically active substances, prodrugs, cytotoxic substances, and diagnostic substances such as fluorochromes, dyes, enzyme substrates, etc., can be coupled a metal complexed with the aryl bidentate isonitrile ligands of the present invention.

The present invention can be used to deliver fluorochromes and vital dyes into cells. Examples of such fluorochromes and vital dyes are well known to those skilled in the art and include, for example, fluorescein, rhodamine, coumarin, indocyanine Cy 5.5, NN382, Texas red, DAPI, EDANS, DABCYL and ethidium bromide.

A wide variety of drugs are suitable for use with the present invention, and include, for example, conventional chemotherapeutics, such as vinblastine, doxorubicin, bleomycin, methotrexate, 5-fluorouricil, 6-thioguanine, cytarabine, cyclophosphamide, taxol, taxotere, cis-platin, adriamycin, mitomycin, and vincristine as well as other conventional chemotherapeutics as described in Cancer: Principles and Practice of Oncology, 5th Ed., V. T. Devita, S. Hellman, S. A. Rosenberg, J. B. Lippincott, Co., Phila, 1997, pp. 3125. Also suitable for use in the present invention are experimental drugs, such as UCN-01, acivicin, 9-aminocamptothecin, azacitidine, bromodeoxyuridine, bryostatin, carboplatin, dideoxyinosine, echinomycin, fazarabine, hepsulfam, homoharringtonine, iododeoxyuridine, leucovorin, merbarone, misonidazole, pentostatin, semustine, suramine, mephthalamidine, teroxirone, triciribine phosphate and trimetrexate as well as others as listed in NCl Investigational Drugs, Pharmaceutical Data 1994, NIH Publications No. 94-2141, revised January 1994.

Other useful drugs include anti-inflammatories such as Celebrex, indomethacin, flurbiprofen, ketoprofen, ibuprofen and phenylbutazone; antibiotics such as beta-lactams, aminoglycosides, macrolides, tetracyclines, pryridonecarboxylic acids and phosphomycin; amino acids such as ascorbic acid and N-acetyltryptophan; antifungal agents; prostaglandins; vitamins; steroids; and antiviral agents such as AZT, DDI, acyclovir, gancyclovir, idoxuridine, amantadine and vidarabine.

Pharmacologically active substances that can be conjugated to the complexes of the present invention include, but are not limited to, enzymes such as transferases, hydrolyses, isomerases, proteases, ligases, kinases, and oxidoreductases such as esterases, phosphatases, glycosidases, and peptidases; enzyme inhibitors such as leupeptin, chymostatin and pepstatin; growth factors; and transcription factors or domains derived from each.

Other substances that can be conjugated to the complexes of the present invention include nucleic acids, such as siRNA or gene therapy agents. The nucleic acids may be condensed with cationic peptides or polymers. The nucleic acids may be comprised of nucleic acid analogs, e.g., PNA.

In addition, the radioactive and non-radioactive metals, pharmacologically active substances, prodrugs, cytotoxic substances, and diagnostic substances used herein may themselves provide target cell, tissue or organ specificity.

The references cited throughout the specification are incorporated herein by reference in their entirety.

### Example

FIG. 1A shows the structure of sestamibi (CARDIOLITE®). FIGS. 1E, 1F and 1G show exemplary aryl bidentate isonitrile ligands of the present invention. FIG. 1H shows a general aryl substitution on a ligand as in FIG. 1E. FIG. 1B shows a triphenyl technetium ion generated with the aryl bidentate isonitrile ligands depicted in FIG. 1E. FIG. 1I shows a potential structure of a triphenyl technetium ion generated with the aryl substituted bidentate isonitrile ligand shown in FIG. 1H.

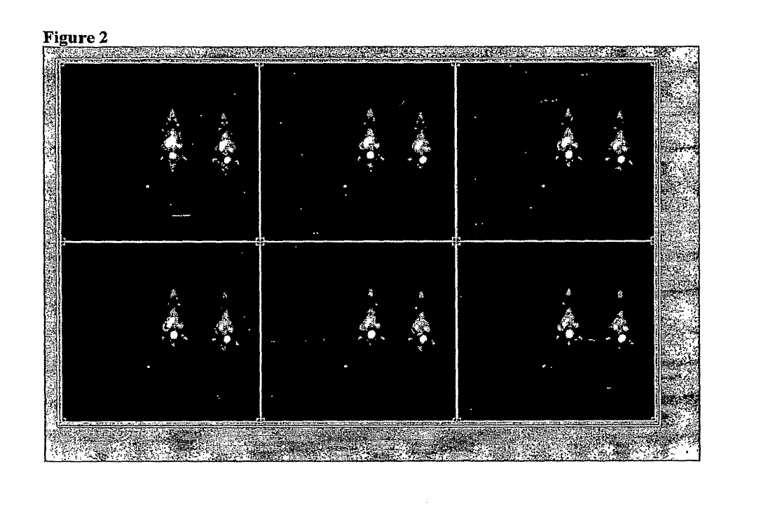

FIG. 2 shows successful imaging in a rat using the Tc-99m complex generated from the aryl bidentate isonitrile ligand in FIG. 1G.

We claim:

1. An aryl bidentate isonitrile ligand having the structure: 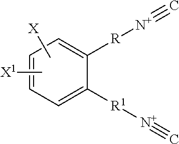
wherein X and X¹ are independently a hydrogen or a substituent on the aryl group and R and R¹ are independently a straight or branched alkyl group.

2. The aryl bidentate isonitrile ligand of claim 1, wherein about 0 to about 4 carbons separate the aryl group from the nitrogen of the CN group.

3. The aryl bidentate isonitrile ligand of claim 1, wherein 3 aryl bidentate isonitrile ligands form a complex with a metal Z.

4. The aryl bidentate isonitrile ligand of claim 3, wherein the metal Z is a radionuclide is Tc-99m or an isotope of rhenium.

5. The aryl bidentate isonitrile ligand of claim 3, wherein the metal is attached to a fluorescent agent.

6. The aryl bidentate isonitrile ligand of claim 3, wherein the metal is attached to a pharmaceutical agent

7. A method for imaging body tissues comprising administering to a subject a radiopharmaceutical composition comprising a coordination complex of an aryl bidentate isonitrile ligand and a radionuclide, and detecting the localization of such complex in the body tissues by a gamma camera.

8. The method of claim 7, wherein the radionuclide is Tc-99m or an isotope of rhenium.

9. The method of claim 7, wherein the body tissue is myocardium.

10. A method for delivering a therapeutic composition to a subject, comprising administering to a subject a therapeutic composition comprising a coordination complex of an aryl bidentate isonitrile ligand and a therapeutic.

11. The method of claim 10, wherein the therapeutic is a radionuclide.

12. The method of claim 10, wherein the aryl bidentate isonitrile ligand is complexed with a metal and the therapeutic is a pharmaceutically active composition covalently attached to the metal.
